(19) United States
(12) Patent Application Publication
(10) Pub. No.: US 2002/0120409 A1
(43) Pub. Date: Aug. 29, 2002

(54) Methods for gene expression analysis

(75) Inventors: Yanxiang Cao, Mountain View, CA (US); Xiaoqiong Chen, San Jose, CA (US); Carsten Rosenow, Redwood City, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/090,320

(22) Filed: Mar. 1, 2002

Related U.S. Application Data
(63) Continuation-in-part of application No. 09/641,081, filed on Aug. 16, 2000.
(60) Provisional application No. 60/205,432, filed on May 19, 2000.

(51) Int. Cl. C12Q 1/68; G06F 19/00; G01N 33/48; G01N 33/50

(52) U.S. Cl. 702/20; 435/6

(57) ABSTRACT

The present invention relates to the detection of nucleic acids, preferably RNA. Primers of random sequence hybridize to the template at regions where complementarity exists between a given random primer and the template. The hybridized primers are used to prime cDNA synthesis. The resulting cDNA product is not biased toward representation of the 3′ ends of the RNAs in the starting sample.

### RELATED APPLICATIONS

The present application claims priority to U.S. application Ser. No. 09/641,081 filed Aug. 16, 2000 the disclosure of which is incorporated herein by reference in its entirety for all purposes.

### FIELD OF THE INVENTION

The present invention relates generally to the field of expression monitoring. More particularly it relates to the field of determining expression of particular genes as reflected by their respective RNA species present in a sample.

### BACKGROUND OF THE INVENTION

Many biological functions are accomplished by altering the expression of various genes through transcriptional (e.g. through control of initiation, provision of RNA precursors, RNA processing, etc.) and/or translational control. For example, fundamental biological processes such as cell cycle progression, cell differentiation and cell death, are often characterized by the variations in the expression levels of a group of genes.

Gene expression is also associated with pathogenesis. For example, the lack of sufficient expression of functional tumor suppressor genes and/or the over expression of oncogenes or protooncogenes could lead to tumorgenesis (see, Marshall, Cell 64: 313-326 (1991); Weinberg, Science 254: 1138-1146 (1991), incorporated herein by reference in their entirety). Thus, changes in the expression levels of particular genes (e.g. oncogenes or tumor suppressors) serve as signposts for the presence and progression of various diseases.

Alternative splicing is one aspect of gene expression that has taken on increased importance following the publication of the human genome (see, Venter et al., Science 291: 1304-1351 (2001) and Lander et al., Nature 409: 860-921 (2001) incorporated herein by reference in their entirety). Recent work indicates that the human genome probably contains fewer genes than anticipated, possibly only 30,000 instead of earlier estimates of more than 90,000. This number of genes is much smaller than expected given the physiological complexity of humans, suggesting that other mechanisms of generating diverse cellular products may take on an increased importance in the future. Alternative splicing is one such mechanism for generating increased complexity. Alternative splicing allows for the production of many different gene products from a single gene and is the major mechanism for generating isoform diversity. It is estimated that at lest 35% of human genes are alternatively spliced (See, Hastings and Krainer, Curr. Op. Cell Bio. 13: 302-309 (2001), incorporated herein by reference in its entirety for all purposes). Detection of alternatively spliced forms of a gene is an important aspect of gene expression analysis.

Analysis of gene expression is often accomplished by making at least one, and often many, labeled copies of the transcripts followed by detection and quantification of the resulting signal. Methods that amplify nucleic acids by extending a primer that is hybridized near the 3′ end of the nucleic acid can result in a preferential amplification of the 3′ end of the nucleic acid compared to the 5′ end of the nucleic acid. This bias toward generating signal from the 3′ end of transcripts can effect quantitative and qualitative analysis of the sample so methods that reduce this bias are needed.

### BRIEF DESCRIPTION OF FIGURES

FIG. 1 shows a schematic of a method of synthesizing labeled cDNA from RNA. Random primers are hybridized to an RNA sample. The primers may hybridize at locations within or near the ends of a RNA. Multiple primers of different sequence may bind at multiple locations within a RNA. The hybridized primers are extended using reverse transcriptase to form cDNA. The resulting cDNA products may be of different lengths and may represent different, but possibly overlapping, regions of a single RNA from the starting material. Following cDNA synthesis the RNA may be removed. The cDNA may then be fragmented and the fragments labeled by end labeling.

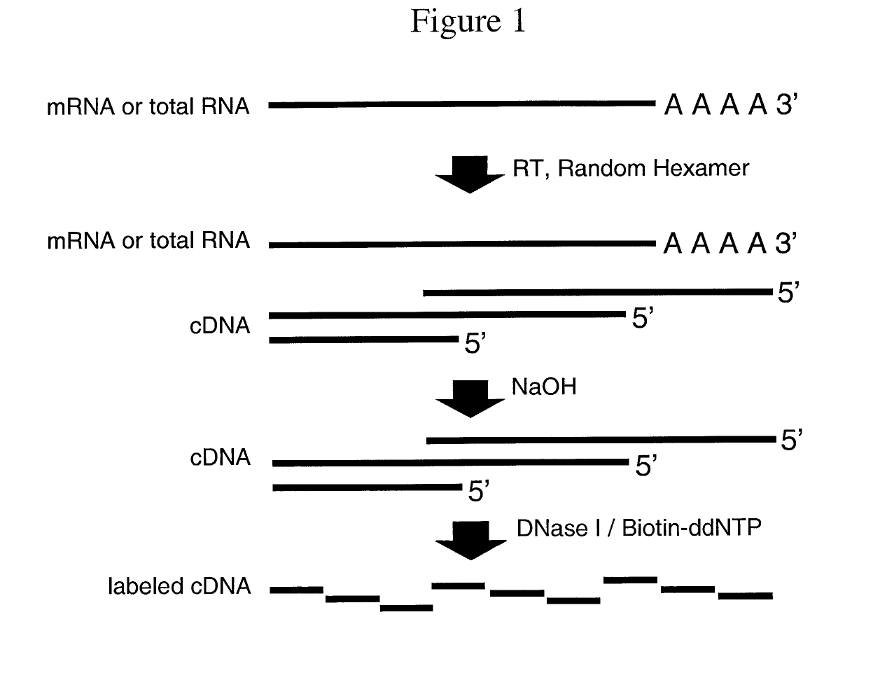

### SUMMARY OF THE INVENTION

The present invention provides a method for the detection of nucleic acids that may comprise synthesizing single-stranded DNA from a RNA population. The present invention also provides a method for preparing a population of cDNA from a population of RNA, preferably the cDNA is detectably labeled. More specifically, the method comprises contacting a RNA population with a collection of random primers; generating a first cDNA strand from the MRNA strand by extending the primers by reverse transcriptase and the appropriate nucleotides under the appropriate conditions, which creates a RNA:DNA duplex; denaturing the RNA:DNA duplex by digesting or degrading the RNA; fragmenting the cDNA and labeling the cDNA fragments. The population of cDNA is representative of the population of RNA in the starting sample.

Among other factors, the present invention provides a method for detection of nucleic acids that is not biased toward detection of the 3′ end of the nucleic acid. The methods of the invention are particularly useful for detection and analysis of RNAs present in multiple isoforms. Additionally, the present invention can be used to detect RNA regardless of polyadenylation.

The present invention also preferably provides methods, which may further comprise contacting the cDNA fragments with a solid support comprising nucleic acid probes, and detecting the presence or absence of hybridization of the cDNA fragments to the nucleic acid probes on the solid support. In a preferred embodiment, the solid support, which may comprise nucleic acid probes, can be selected from the group consisting of a nucleic acid probe array, a membrane blot, a microwell, a bead, and a sample tube.

In yet another preferred embodiment, the invention relates to a kit comprising reagents and instructions for the detection of RNA. Preferably, the kit includes a reaction vessel containing one or more reagents in concentrated form, where the reagent may be an enzyme or enzyme mixture. The kit also includes a container, instructions for use, random primers, reverse transcriptase, terminal transferase, labeled nucleotides and a nucleic acid probe array.

In another embodiment the invention relates to the detection of one or more isoforms in an RNA sample.

In another embodiment the invention relates to a method to detect and distinguish between different isoforms present in an RNA sample.

### DETAILED DESCRIPTION OF THE PREFERRED EMBODIMENTS

General

The present invention relies on many patents, applications and other references for details known to those of the art. Therefore, when a patent, application, or other reference is cited or repeated below, it should be understood that it is incorporated by reference in its entirety for all purposes as well as for the proposition that is recited.

As used in the specification and claims, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, fungi, bacteria, or cells derived from any of the above.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example hereinbelow. However, other equivalent conventional procedures can, of course, also be used. Such conventional techniques can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), all of which are herein incorporated in their entirety by reference for all purposes.

Methods and techniques applicable to array synthesis have been described in U.S. Pat. Nos. 5,143,854, 5,242,974, 5,252,743, 5,324,633, 5,384,261, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555 and 6,309,823, which are all incorporated herein by reference in their entirety for all purposes.

Additionally, gene expression monitoring and sample preparation methods can be shown in U.S. Pat. Nos. 5,800,992, 6,040,138, 6,013,449, 6,303,301, and 6,308,170.

Definitions

Nucleic acids according to the present invention may include any polymer or oligomer of pyrimidine and purine bases, preferably cytosine, thymine, and uracil, and adenine and guanine, respectively. See, Nelson and Cox (2000), Lehninger, Principles of Biochemistry 3^{rd} Ed., W. H. Freeman Pub., New York, NY and Berg et al. (2002) Biochemistry, 5^{th} Ed., W. H. Freeman Pub., New York, N.Y., both of which are incorporated herein by reference. Indeed, the present invention contemplates any deoxyribonucleotide, ribonucleotide or peptide nucleic acid component, and any chemical variants or analogs thereof, such as methylated, hydroxymethylated or glycosylated forms of these bases, and the like. (See, U.S. Pat. No. 6,156,501 which is incorporated herein by reference in its entirety.) The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. Oligonucleotide and polynucleotide are included in this definition and relate to two or more nucleic acids in a polynucleotide.

Random primers are a mixture of oligodeoxyribonucleotides of variable sequence which may be used for priming cDNA synthesis at many different locations in a nucleic acid sample. Random primers are a collection of many different species of primers, each species having a different sequence. For example, random hexamers are in the form 5′-d(N6)-3′ where N is any base. The ratio of different species in the random primers may be random, approximately equal or it may be varied. For example, one or more species may be included in the mixture at a higher level than other species. In the current invention random primers may be mixed with other species of primers, for example, in one preferred embodiment random primers are mixed with oligo dT. Random primers of many different lengths can be used in the current invention. In the current invention random primers are preferably not less than 5, 6, 7, 8, 9, or 12 nucleotides in length and not longer than 8, 9, 12, 15, 24 or 36 nucleotides in length. Random primers for use in the present invention can be custom made or purchased from a variety of commercial sources, for example, New England Biolabs, Beverly, Mass.

Isoform or mRNA isoform: A single gene may give rise to more than one mRNA sequence differing in the precise combination of exon sequences or 5′ or 3′ sequences, which are called isoforms or mRNA isoforms. Isoforms may result from alternative transcriptional events such as the use of alternative promoters. Many genes are known to have several alternative promoters, the use of each promoter resulting in one particular species of transcript. Generally, the use of a relatively 5′ promoter results in a product that has additional sequence elements that are absent in the products transcribed from relatively 3′ promoters. The use of alternative promoters is frequently employed to regulate tissue specific gene expression. For example, the human dystrophin gene has at least seven promoters. The most 5′ upstream promoter is used to transcribe a brain specific transcript; a promoter 100 kb down-stream from the first promoter is used to transcribe a muscle specific transcript and a promoter 100 kb downstream of the second promoter is used to transcribe a Purkinje cell specific transcript.

A gene may be transcribed to form a single species of pre-RNA that may be processed, by alternative splicing, into multiple isoforms differing in their precise combination of exon sequences. Alternative splicing can expand the coding capacity of a single gene to allow production of many different protein isoforms, often having different functions. Often these isoforms are expressed in a tissue or temporal specific manner. The Drosophila Dscam gene provides a striking example of how diversity can be generated by alternative splicing. Dscam has 24 exons, with 12 alternative versions of exon 4, 48 versions of exon 6, 33 versions of exon 9 and 2 versions of exon 17. The combinatorial use of alternative exons in the Dscam premRNA can potentially generate 38,016 different protein isoforms (See, Hastings and Krainer, (2001) Curr. Op. Cell Bio. 13: 302-309, which is incorporated herein by reference).

In addition to alternative splicing, other processing events such as RNA editing or the use of alternative polyadenylation sites can result in the formation of different RNA isoforms from a single gene or pre-RNA. Isoforms may also result from combinations of processing events.

Mutations in alternatively or constitutively spliced genes can also trigger aberrant splicing, which can lead to human disease. For example, mutations in Wilm's tumor-suppressor gene 1 result in misregulation of alternative splicing and the production of an aberrant WT1 gene product which is associated with childhood kidney tumors (see, Grabowski and Black, (2001), Prog. In Neurobiol. 65: 289-308, incorporated herein by reference in its entirety for all purposes). Mutations can, for example, result in the exclusion of an exon that is normally included in the final product, aberrant splice site selection within an exon or an intron or other splicing errors that result in aberrant inclusion or exclusion of sequence.

Array: An array comprises a support, preferably solid, with nucleic acid probes attached to said support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., Science, 251:767-777 (1991), each of which is incorporated by reference in its entirety for all purposes. These arrays may generally be produced using mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. No. 5,384,261, 6,040,193, and 6,121,048 which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992, which are hereby incorporated by reference in their entirety for all purposes.)

Arrays may be packaged in such a manner as to allow for diagnostics or can be an all-inclusive device; e.g., U.S. Pat. Nos. 5,856,174 and 5,922,591 incorporated in their entirety by reference for all purposes. (See also U.S. patent application Ser. No. 09/545,207 for additional information concerning arrays, their manufacture, and their characteristics.) It is hereby incorporated by reference in its entirety for all purposes.

Preferred arrays are commercially available from Affymetrix under the brand name GeneChip® and are directed to a variety of purposes, including gene expression monitoring for a variety of eukaryotic and prokaryotic species. (See Affymetrix Inc., Santa Clara and their website at affymetrix.com.)

The Process

In general, the presently preferred invention enables a user to make labeled cDNA from RNA for gene expression monitoring experiments. Although one of skill in the art will recognize that other uses may be made of the labeled cDNA. An overview of the process is as follows. RNA is contacted with a collection of random primers. Hybridized primers are extended with reverse transcriptase to make cDNA. The RNA strand is then separated from the cDNA by, for example, digestion with RNase or heat or base hydrolysis.

More specifically, the presently preferred invention is as follows: RNA is annealed with random primers creating a primer-template mixture. Hybridization conditions are well known in the art, (see, for example, Sambrook and Russell Chapter 9, Protocol 7 and Ausubel et al. eds., 1993 Current Protocols in Molecular Biology, John Wiley and Sons, New York, N.Y.), but may involve a step of denaturation to disrupt base pairing interactions followed by incubation under conditions that will favor hybridization. Hybridization is primarily influenced by four parameters: temperature, pH, concentration of monovalent cations and presence or organic solvents. The ideal temperature for hybridization is dependent on the melting point (Tₘ) of the hybrid, which is dependent on the length and sequence of the primers. One approach for hybridization is to mix the primers with the nucleic acid sample and heat the mixture to a temperature that will denature the sample then to slowly cool the mixture to a temperature below the Tₘ. The pH for hybridization conditions may be in the range of pH 5 to 9, commonly a pH between 6.5 and 7.5 is used in combination with a buffer containing 20 to 50 mM phosphate. Variation in the salt concentration also affects hybridization, with higher salt concentrations increasing the stability of hybridization. Salt concentrations may be, for example, between 0.1 to 0.3 M. Organic solvents can also be added at varying concentrations, for example, 25% formamide may be added. Primer concentration also impacts hybridization, the higher the concentration of primer the higher the rate of annealing. cDNA synthesis is accomplished by combining the first strand cDNA reagent mix (Superscript II buffer, DTT, and dNTPs) and SuperScriptII with the primer-template mixture and incubating at the appropriate time and temperature. RNA is removed by adding RNase followed by incubation at the appropriate time and temperature. Alternatively the RNA can be removed by incubation in NaOH followed by neutralization with HCl. The cDNA is then purified and fragmented by, for example, incubation with DNase. Finally, the cDNA fragments are labeled by, for example, incubation with terminal transferase and the appropriate labeled nucleotides, yielding labeled cDNA fragments.

Those skilled in the art will recognize that the products and methods embodied in the present invention may be applied to a variety of systems, including commercially available gene expression monitoring systems involving nucleic acid probe arrays, membrane blots, microwells, beads, and sample tubes, constructed with various materials using various methods known in the art. Accordingly, the present invention is not limited to any particular environment, and the following description of specific embodiments of the present invention are for illustrative purposes only.

In a preferred embodiment, RNA is used as a template for the production of the labeled cDNA of the present invention. However, other nucleic acids may be used as starting material. For example, DNA or oligonucleotides may be used as template for cDNA synthesis.

The reaction vessel according to the present invention may include a membrane, filter, microscope slide, microwell, sample tube, array, or the like. (See International Patent applications No. PCT/US95/07377 and PCTIUS96/11147, which are expressly incorporated herein by reference.) The reaction vessel may be made of various materials, including polystyrene, polycarbonate, plastics, glass, ceramic, stainless steel, or the like. The reaction vessel may preferably have a rigid or semi-rigid surface, and may preferably be conical (e.g., sample tube) or substantially planar (e.g., flat surface) with appropriate wells, raised regions, etched trenches, or the like. The reaction vessel may also include a gel or matrix in which nucleic acids may be embedded. (See A. Mirzabekov et al., Anal. Biochem. 259(1):34-41 (1998), and U.S. Pat. No. 5,744,305 both of which are expressly incorporated herein by reference.)

The single-stranded or double-stranded nucleic acid populations according to the present invention may refer to any mixture of two or more distinct species of single-stranded RNA, DNA or double-stranded DNA, which may include DNA representing genomic DNA, genes, gene fragments, oligonucleotides, polynucleotides, nucleic acids, PCR products, expressed sequence tags (ESTs), or nucleotide sequences corresponding to known or suspected single nucleotide polymorphisms (SNPs), having nucleotide sequences that may overlap in part or not at all when compared to one another. The species may be distinct based on any chemical or biological differences, including differences in base composition, order, length, or conformation. The single-stranded nucleic acid population may be isolated or produced according to methods known in the art, and may include single-stranded cDNA produced from a MRNA template, single-stranded DNA isolated from double-stranded DNA, or single-stranded DNA synthesized as an oligonucleotide. The double-stranded DNA population may also be isolated according to methods known in the art, such as PCR, reverse transcription, and the like.

Where the nucleic acid sample contains RNA, the RNA may be total RNA, poly(A)⁺ RNA, mRNA, rRNA, or tRNA, and may be isolated according to methods known in the art. (See, e.g., Sambrook and Russell, (2001) Molecular Cloning: A Laboratory Manual, Cold Spring Harbor Laboratory Press, Cold Spring Harbor, N.Y., which is expressly incorporated herein by reference.) The RNA may be heterogeneous, referring to any mixture of two or more distinct species of RNA. The species may be distinct based on any chemical or biological differences, including differences in base composition, length, or conformation. The RNA may contain full length mRNAs or mRNA fragments (i.e., less than full length) resulting from in vivo, in situ, or in vitro transcriptional events involving corresponding genes, gene fragments, or other DNA templates. In a preferred embodiment, the RNA population of the present invention may contain single-stranded poly(A)+RNA, which may be obtained from an RNA mixture (e.g., a whole cell RNA preparation), for example, by affinity chromatography purification through an oligo-dT cellulose column.

Methods of isolating total RNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993), which is incorporated herein by reference in its entirety for all purposes.

In a preferred embodiment, the total RNA is isolated from a given sample using, for example, an acid guanidinium-phenol-chloroform extraction method and polyA⁺ MRNA is isolated by oligo dT column chromatography or by using (dT)n magnetic beads. (See e.g., Sambrook and Russell, (2001) Molecular Cloning: A Laboratory Manual, 3^{rd} Ed., Cold Spring Harbor Laboratory Press, Cold Spring Harbor, N.Y., or F. Ausubel et al., ed. (1993) Current Protocols in Molecular Biology John Wiley and Sons, New York, N.Y.). (See also PCT/US99/25200 for complexity management and other sample preparation techniques, which is hereby incorporated by reference in its entirety for all purposes.)

The cDNA of the present invention may be produced according to methods known in the art. (See, e.g, Sambrook and Russell (2001)). In a preferred embodiment, a sample population of RNA may be used to produce corresponding cDNA in the presence of reverse transcriptase, random primers and dNTPs. Reverse transcriptase may be any enzyme that is capable of synthesizing a corresponding cDNA from an RNA template in the presence of the appropriate primers and nucleoside triphosphates. In a preferred embodiment, the reverse transcriptase may be from avian myeloblastosis virus (AMV), Moloney murine leukemia virus (MMuLV) or Rous Sarcoma Virus (RSV), for example, and may be a thermal stable enzyme (e.g., rTth DNA polymerase available from Applied Biosystems, Foster City, Calif.).

In a preferred embodiment of the present invention, the single-stranded cDNA produced using an RNA population as template may be separated from any resulting RNA templates by degradation of the RNA using heat, chemical (e.g. high pH) or enzyme treatment (e.g., RNase H or RNase A). In a preferred embodiment, terminal transferase (TdT) may be used to add sequences to the 3′-termini of the single-stranded DNA. Terminal transferase catalyzes the addition of mononucleotides from dNTPs to the 3′ terminus of nucleic acids. Terminal transferase can be used to add a string of nucleotides to the 3′ end of a single or double stranded nucleic acid. In a preferred embodiment at least some of the added nucleotides are labeled. In a preferred embodiment a homopolymer tail is added. The length and distribution of the homopolymer tails added by TdT depends on several factors including the nucleotide used, substrate concentrations, ratio of DNA to nucleotide, and reaction time and temperature. For a discussion of factors affecting the length and distribution of homopolymer tails generated by TdT see, Eun, H-M. (1996) Enzymology Primer for Recombinant DNA Technology, Academic Press, Inc., San Diego, Calif., which is herein incorporated by reference,.

Reverse transcriptase (e.g., either derived from AMV or MuLV) is available from a large number of commercial sources including Invitrogen/LTI, Amersham Phamacia Biotech (APB)/USB, Qiagen, and others. Other enzymes required or desired are also available from these vendors among others, such as Promega, and Epicentre. Nucleotides such as DNTPS, unique nucleotide sequences, and β-NAD are available from a variety of commercial sources such as APB, Roche Biochemicals, and Sigma Chemicals. Buffers, salts and cofactors required or desired for these reactions can usually be purchased from the vendor that supplies a respective enzyme or assembled from materials commonly available, e.g., from Sigma Chemical.

In a preferred embodiment of the present invention, the cDNA may be labeled by the incorporation of biotinylated, fluorescently labeled or radiolabeled dNTPs, or other compounds containing labeling compounds. The labeling of a nucleic acid is typically performed by covalently attaching a detectable group (label) to either an internal or terminal position. In the present invention labeling compounds may be incorporated, for example, by 3′ end labeling with terminal transferase, by labeling the primer or by incorporation of labeled compounds during cDNA synthesis. Scientists have reported a number of detectable nucleotide analogues that have been enzymatically incorporated into an oligo- or polynucleotide. Langer et al., for example, disclosed analogues of dUTP and UTP that contain a covalently bound biotin moiety. See, (1981) Proc. Natl. Acad. Sci. USA, 78: 6633-6637.

In one preferred embodiment of the current invention the cDNA is labeled by incorporation of any nucleotide analog that can be incorporated into the cDNA by a polymerase. Nucleotide analogs such as those described in U.S. patent application Ser. No. 09/952,387, which is incorporated herein by reference in its entirety for all purposes, may be used in one embodiment of the invention. These include heterocyclic derivatives containing a detectable moiety and are of the following structure:

A—O—CH₂—T—H_{c}—L—(M)ₘ—Q

wherein A is hydrogen or a functional group that permits the attachment of the nucleic acid labeling compound to a nucleic acid; T is a template moiety; H_{c} is a heterocyclic group; L is a linker moiety; Q is a detectable moiety; and M is a connecting group, wherein m is an integer ranging from 0 to about 5. One such derivative which is particularly preferred in one embodiment of the invention is bio-v-dNTPs.

In a preferred embodiment of the present invention, the detectable label may be radioactive, fluorometric, enzymatic, or colorimetric, or a substrate for detection (e.g., biotin). When biotin labeled nucleotides are used labeled avidin can subsequently be bound to the biotin-labeled polynucleotides. The labeled avidin may contain any desirable detectable label. Other detection methods, involving characteristics such as scattering, IR, polarization, mass, and charge changes, may also be within the scope of the present invention. Methods of labeling are well known in the art. (See, for example, Sambrook and Russell, (2001) Molecular Cloning: A Laboratory Manual, 3^{rd} Ed., Cold Spring Harbor Laboratory Press, Cold Spring Harbor, N.Y.).

In another preferred embodiment of the present invention the cDNA is amplified according to methods known in the art. Methods may include, for example, polymerase chain reaction, (see, e.g. U.S. Pat. Nos. 4,683,195 and 4,683,202). Other amplification methods include the ligase chain reaction (LCR) e.g., Wu and Wallace, Genomics 4, 560 (1989) and Landegren et al., Science 241, 1077 (1988), Burg, U.S. Pat. Nos. 5,437,990, 5,215,899, 5,466,586, 4,357,421, Gubler et al., 1985, Biochemica et Biophysica Acta, Displacement Synthesis of Globin Complementary DNA: Evidence for Sequence Amplification, transcription amplification, Kwoh et al., Proc. Natl. Acad. Sci. USA 86, 1173 (1989), self-sustained sequence replication, Guatelli et al., Proc. Nat. Acad. Sci. USA, 87, 1874 (1990) and WO 88/10315 and WO 90/06995 and nucleic acid based sequence amplification (NABSA). The latter two amplification methods include isothermal reactions based on isothermal transcription, which produce both single-stranded RNA (ssRNA) and double-stranded DNA (dsDNA) as the amplification products in a ratio of about 30 or 100 to 1, respectively. Second strand priming can occur by hairpin loop formation, RNAse H digestion products, and the 3′ end of any nucleic acid present in a reaction capable of forming an extensible complex with the first strand DNA.

In another preferred embodiment of the present invention the signal is amplified according to methods known in the art which may include, for example, tyramide signal amplification (TSA), (see, U.S. Pat. Nos. 5,196,306, 5,583,001, 5,731,158 and EP patent no. 0465 577 B1 which are herein incorporated by reference), dendrimer signal amplification (see, U.S. Pat. Nos., 5,487,973, 6,203,989, 6,261,779 and 6,274,723 which are incorporated by reference herein), rolling circle amplification (see, U.S. Pat. Nos. 6,210,884 and 6,183,960 which are incorporated herein by reference) or any other mechanism of signal amplification, (see, U.S. Pat. No. 6,203,989 which is incorporated herein by reference).

In a particularly preferred embodiment of the present invention, the signal detected using a probe located near the 3′ end of an RNA species in the starting material does not exceed the signal detected using a probe located near the 5′ end of that RNA species by more than 2 fold.

In a preferred embodiment, the cDNA of the present invention may be analyzed with a gene expression monitoring system. Several such systems are known. (See, e.g., U.S. Pat. No. 5,677,195; Wodicka et al., Nature Biotechnology 15:1359-1367 (1997); Lockhart et al., Nature Biotechnology 14:1675-1680 (1996), which are expressly incorporated herein by reference.) A preferred gene expression monitoring system according to the present invention may be a nucleic acid probe array, such as the GeneChip® nucleic acid probe array (Affymetrix, Santa Clara, Calif.). (See, U.S. Pat. Nos. 5,744,305, 5,445,934, 5,800,992, 6,040,193 and International Patent applications PCT/US95/07377, PCT/US96/14839, and PCT/US96/14839, which are expressly incorporated herein by reference.) A nucleic acid probe array preferably comprises nucleic acids bound to a substrate in known locations. In other embodiments, the system may include a solid support or substrate, such as a membrane, filter, microscope slide, microwell, sample tube, bead, bead array, or the like. The solid support may be made of various materials, including paper, cellulose, gel, nylon, polystyrene, polycarbonate, plastics, glass, ceramic, stainless steel, or the like including any other support cited in U.S. Pat. Nos. 5,744,305, 5,800,992, 6,309,822 or 6,040,193. The solid support may preferably have a rigid or semi-rigid surface, and may preferably be spherical (e.g., bead) or substantially planar (e.g., flat surface) with appropriate wells, raised regions, etched trenches, or the like. The solid support may also include a gel or matrix in which nucleic acids may be embedded. The gene expression monitoring system, in a preferred embodiment, may comprise a nucleic acid probe array (including an oligonucleotide array, a cDNA array, a spotted array, and the like), membrane blot (such as used in hybridization analysis such as Northern, Southern, dot, and the like), or microwells, sample tubes, beads or fibers (or any solid support comprising bound nucleic acids). See U.S. Pat. Nos. 5,770,722, 5,744,305, 5,677,195 5,445,934, and 6,040,193 which are incorporated here in their entirety by reference. (See also Examples, infra.) The gene expression monitoring system may also comprise nucleic acid probes in solution.

Preferred high density arrays for gene expression analysis and genotyping comprise greater than about 100, preferably greater than about 1000, more preferably greater than about 16,000 and most preferably greater than 65,000 or 250,000 or even greater than about 1,000,000 different oligonucleotide probes, preferably in less than 1 cm² of surface area. The oligonucleotide probes range from about 5, 10, or 15 to about 50 or about 500 nucleotides, more preferably from about 10 to about 30, 40 or 50 nucleotides and most preferably from about 15 to about 30,40 or 50 nucleotides in length.

Oligonucleotide probe arrays containing probes targeting exon sequences may be selected to detect and quantify various transcripts. By using these exon probes, the presence of particular isoforms in a biological sample may be determined. Probes may also be targeted to detect regions shared by one or more isoforms or to regions that are present in only a subset of isoforms.

The gene expression monitoring system according to the present invention may be used to facilitate a comparative analysis of expression in different cells or tissues, different subpopulations of the same cells or tissues, different physiological states of the same cells or tissue, different developmental stages of the same cells or tissue, or different cell populations of the same tissue. (See U.S. Pat. Nos. 5,800,922, 6,040,138 and 6,309,822.)

The methods of the present invention may be used, for example, to simultaneously detect multiple RNA isoforms resulting from a single gene, separately detect all RNA isoforms resulting from a single gene, and to identify changes in the ratios of splice variants. In many of the preferred embodiments, the unbiased detection methods of the present invention can provide reproducible results (i.e., within statistically significant margins of error or degrees of confidence) sufficient to facilitate the measurement of quantitative as well as qualitative differences in the tested samples.

The detection methods of the present invention may also facilitate the identification of single nucleotide polymorphisms (SNPs) (i.e., point mutations that can serve, for example, as markers in the study of genetically inherited diseases) and other genotyping methods. (See e.g., Collins et al., 282 Science 682 (1998), which is expressly incorporated herein by reference.) The mapping of SNPs can occur by any of various methods known in the art, one such method being described in U.S. Pat. No. 5,679,524, which is hereby incorporated by reference. (See also, U.S. Pat. Nos. 5,547,839, 5,925,525, and 5,968,740 which are hereby incorporated by reference in their entireties.)

The RNA population of the present invention may be obtained or derived from any tissue or cell source. Indeed, the nucleic acid sought to be detected may be obtained from any biological or environmental source, including plant, virion, bacteria, fungi, or algae, from any sample, including body fluid or soil. In one embodiment, eukaryotic tissue is preferred, and in another, mammalian tissue is preferred, and in yet another, human tissue is preferred. The tissue or cell source may include a tissue biopsy sample, a cell sorted population, cell culture, or a single cell. In a preferred embodiment, the tissue source may include brain, liver, heart, kidney, lung, spleen, retina, bone, lymph node, endocrine gland, reproductive organ, blood, nerve, vascular tissue, and olfactory epithelium. In yet another preferred embodiment, the tissue or cell source may be embryonic or tumorigenic.

Tumorigenic tissue according to the present invention may include tissue associated with malignant and pre-neoplastic conditions, not limited to the following: acute lymphocytic leukemia, acute myelocytic leukemia, myeloblastic leukemia, promyelocytic leukemia, myelomonocytic leukemia, monocytic leukemia, erythroleukemia, chronic myelocytic (granulocytic) leukemia, chronic lymphocytic leukemia, polycythemia vera, lymphoma, Hodgkin's disease, non-Hodgkin's disease, multiple myeloma, Waldenstrom's macroglobulinemia, heavy chain disease, solid tumors, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. (See Fishman et al., Medicine, 2d Ed. (J. B. Lippincott Co., Philadelphia, Pa. 1985), which is expressly incorporated herein by reference.)

In yet another preferred embodiment of the present invention, the cDNA, or fragments thereof, may be immobilized directly or indirectly to a solid support or substrate by methods known in the art (e.g., by chemical or photoreactive interaction, or a combination thereof). (See U.S. Pat. Nos. 5,800,992, 6,040,138 and 6,040,193.) The resulting immobilized nucleic acid may be used as probes to detect nucleic acids in a sample population that can hybridize under desired stringency conditions. Such nucleic acids may include DNA contained in the clones and vectors of cDNA libraries.

The materials for use in the present invention are ideally suited for the preparation of a kit suitable for detection of nucleic acids. Such a kit may comprise reaction vessels, each with one or more of the various reagents, preferably in concentrated form, utilized in the methods. The reagents may comprise, but are not limited to the following: buffer, appropriate nucleotide triphosphates (e.g. dATP, dCTP, dGTP, dTTP; or dUTP) reverse transcriptase, RNase H, RNase A, terminal transferase, a mixture of random primers, a mixture of random primers and oligo d(T), labeled nucleotide triphosphates, and labeled nucleotide analogs. In addition, the reaction vessels in the kit may comprise 0.2-1.5 ml tubes capable of fitting a standard thermocycler, which may be available singly, in strips of 8, 12, 24, 48, or 96 well plates depending on the quantity of reactions desired. Hence, the reactions may be automated, e.g., performed in a PCR theromcycler. The thermocyclers may include, but are not limited to the following: Perkin Elmer 9600, MJ Research PTC 200, Techne Gene E, Erichrom, and Whatman Biometra Ti Thermocycler.

Also, the automated machine of the present invention may include an integrated reaction device and a robotic delivery system. In such cases, part of all of the operation steps may automatically be done in an automated cartridge. (See U.S. Pat. Nos. 5,856,174, 5,922,591, and 6,043,080.)

Without further elaboration, one skilled in the art with the preceding description can utilize the present invention to its fullest extent. The following examples are illustrative only, and not intended to limit the remainder of the disclosure in any way.

### EXAMPLE ONE

Step 1: cDNA Synthesis

Mix 5-10 μg total RNA, as little as 2 μg may be used, with 750 ng random primers in a final volume of 30 μl. Incubate the mixture at 70° C. for 10 min then chill at 4° C. Prepare a mixture of the following: 12 μl 5× 1^{st} strand buffer, 6 μl 100 mM DTT, 3 μl 10 mM dNTP mix, 1.5 μl RNase Inhibitor (Amersham, Piscataway, N.J.) and 7.5 μl Superscript II (Promega, Madison, Wis.) and add to the reaction, bringing the total volume to 60 μl. Incubate at 25° C. for 10 min, 37° C. for 60 min followed by incubation at 42° C. for 60 min. Inactivate the enzyme by incubation at 70° C. for 10 min then hold at 4° C.

Step 2: Removal of RNA and cDNA Purification

Add 20 μl 1N NaOH and incubate at 65° C. for 30 min. Add 20 μl 1N HCL to neutralize. Alternatively RNA may be removed by incubation with RNase. Purify the cDNA using the RNeasy kit (Qiagen, Valencia, Calif.).

Step 3: cDNA Fragmentation

Add 0.8 units DNase I (Promega, Madison, Wis.) per μg of cDNA, in 1× One-Phor-All buffer (Amersham Pharmacia Biotech, Piscataway, N.J.) and incubate at 37° C. for 15 min followed by incubation at 95° C. for 20 min. The length of the resulting fragments is preferably 30 to 150 bases.

Step 4: Labeling

Add 5 μl of 10× TdT buffer, 5 μl 10× CoCl₂, 3 μl terminal transferase (25 U/μl)(NEB, Beverly, Mass.) and 1 μl 1 mM bio-ddATP to the fragmented cDNA. Bring total volume to 50 μl with water and incubate at 37° C. for 1 hour, followed by heat inactivation of enzyme at 95° C. for 15 min. Alternatively add 4 μl rTdT (Roche), 5 μl 5× buffer 7.5 μl 10× CoCl₂ and 2 μl 1 mM bio-v-NTP to the fragmented cDNA (for a description of bio-v-NTP see, US patent application Ser. No. 09/952,387 bring total volume to 50 μl and incubate as above.

Step 5: Hybridize Labeled cDNA to an array.

Mix labeled cDNA with 2× MES, add 2 μl 50 mg/ml BSA (Sigma), 2 μl 10 mg/ml Herring Sperm DNA (Gibco/Invitrogen) and 1 μl Affy Oligo B. Hybridize to array at 45° C. over night.

Once the probe array has been hybridized, stained, and washed, it is scanned and the data is analyzed using GeneChip® software. The areas of hybridization are inputted into a computer and translated into information as to which nucleic acid sequences were present in the original sample. (See, PCT/US00/20563, which is incorporated herein by reference.)

### METHODS OF USE

The current invention is particularly useful for detection of RNAs that are present in multiple distinct forms. RNA processing events such as alternative splicing allow a single species of pre-mRNA to be processed into multiple mRNA isoforms differing in their precise combination of sequence information.

Probes may be designed to take advantage of differences and similarities between isoforms. For example, to detect all species of RNAs resulting from a single species of pre-mRNA, probes may be designed to recognize regions that are common to all isoforms. Likewise, to distinguish between different isoforms probes may be designed to regions that are present in one isoform and absent in another. For example, if a first isoform contains exons A, B and C and a second isoform contains exons A, C and D, to detect both isoforms probes can be designed to hybridize to sequences in exon A and/or C. To detect only the first isoform probes can be designed to hybridize to sequences in exon B. To detect only the second isoform probes can be designed to hybridize to sequences in exon D.

In addition to alternative splicing, other processing steps such as the use of alternative polyadenylation sites can result in distinct isoforms. When using an amplification method that preferentially amplifies only the 3′ end of mRNA, alternative polyadenylation signals can result in poor detection of some transcripts. If, for example, two polyadenylation signals are separated by 1 Kb, a probe that is designed to hybridize to transcripts using the upstream site will not efficiently detect transcripts using the downstream site because the region of probe hybridization will be inefficiently amplified.

The labeled cDNA of the current invention represents each region of the starting RNA approximately evenly. As a result, probes can be designed to all regions of the transcript and are not limited by amplification bias, allowing for detection of multiple isoforms independent of the location of the variation.

Using the current invention probes can be designed to any region of a nucleic acid to be analyzed. It is not necessary to design probes to the 3′ end of the nucleic acid to be analyzed. In addition incorrect information about the actual 3′ end of a nucleic acid will have less impact on detection using the current method. When using a labeling method that has a 3′ bias probes are typically designed to be near the 3′ end in order to insure maximum signal, because the 5′ end may be underrepresented in the labeled sample. If the predicted 3′ end is far from the actual 3′ end this can result in reduced or absent signal.

Detecting distinct isoforms is more difficult with amplification methods that show an amplification bias. For example, if the only difference between a first and second isoform is the inclusion of an exon at the 5′ end of a long MRNA it will be very difficult to distinguish between the two isoforms using an amplification method that is biased toward the 3′ end, especially for longer transcripts. The 5′ end of the transcripts will be poorly represented in the amplified material and probes designed to detect the 5′ exon will provide little or no signal.

The current invention is also useful for detecting the presence or absence of transcription from a region of interest in a genome. Mapping the transcriptionally active regions of a genome can be done by a combination of different complementary methods. One such method is to detect the presence of all transcripts present in a sample by hybridizing a labeled sample that is representative of the sample to an array that has probes to interrogate sequences in a region of interest. (See, U.S. Provisional application 60/339,655 the entire disclosure of which is incorporated herein by reference). The present invention is useful for synthesizing a labeled cDNA sample that is representative of the transcripts. The labeled cDNA may then be hybridized to an array.

1. A method of analyzing an RNA sample comprising:
contacting an RNA sample with random primers under hybridization conditions;
generating cDNA from the RNA sample by extending the random primers with reverse transcriptase to produce cDNA;
degrading the RNA population;
fragmenting the cDNA;
labeling the cDNA fragments;
contacting the labeled cDNA fragments with a solid support comprising nucleic acid probes under hybridization conditions; and
detecting the presence or absence of hybridization of the labeled cDNA fragments to the nucleic acid probes on the solid support.

2. The method of claim 1 wherein, for the majority of RNAs in the starting sample, the number of cDNA copies of a given sequence near the 3′ end of a single species of RNA is not more than twice the number of cDNA copies of a given sequence near the 5′ end of said single species of RNA.

3. The method of claim 1 wherein said RNA is selected from the group consisting of total RNA, mRNA and poly(A)⁺RNA.

4. The method of claim 1 wherein hybridization is detected by detecting a signal from labeled DNA which is hybridized to the solid support.

5. The method of claim 1 wherein the cDNA fragments are labeled by the addition of at least one labeled nucleotide using terminal transferase.

6. The method of claim 4 wherein the signal is amplified.

7. The method of claim 4 wherein the amount of signal detected with a probe to a 3′ region of an RNA from the starting material is not more than twice the amount of signal detected with a probe to a 5′ region of said RNA from the starting material.

8. The method of claim 6 wherein the amount of signal detected with a probe to a 3′ region of an RNA from the starting material is not more than twice the amount of signal detected with a probe to a 5′ region of said RNA from the starting material.

9. The method of claim 1 wherein the molar amount of cDNA fragments that hybridize to a probe to a 3′ region of a RNA and the molar amount of cDNA fragments that hybridize to a probe to a 5′ region of said RNA vary by 2 fold or less.

10. The method of claim 1 wherein the solid support comprising nucleic acid probes is selected from the group consisting of a nucleic acid probe array, a membrane blot, a microwell, a bead, and a sample tube.

11. The method of claim 1 wherein the random primers are 6 nucleotides in length.

12. The method of claim 1 wherein the random primers are 9 nucleotides in length.

13. The method of claim 1 wherein the random primers are 15 nucleotides in length.

14. The method of claim 1 wherein the RNA sample is isolated from a prokaryotic cell.

15. The method of claim 1 wherein the RNA sample is isolated from a eukaryotic cell or tissue.

16. The method of claim 15 wherein the eukaryotic cell or tissue is mammalian.

17. The method of claim 16 wherein the eukaryotic cell or tissue is human.

18. The method of claim 1 wherein the RNA sample is isolated from a source selected from the group consisting of dissected tissue, microdissected tissue, a tissue subregion, a tissue biopsy sample, a cell sorted population, a cell culture, and a single cell.

19. The method of claim 1 wherein the RNA sample is isolated from a cell or tissue source selected from the group consisting of brain, liver, heart, kidney, lung, retina, bone, lymph node, endocrine gland, reproductive organ, blood, nerve, vascular tissue, and olfactory epithelium.

20. The method of claim 1 wherein the RNA sample is isolated from a cell or tissue source selected from the group consisting of embryonic and tumorigenic.

21. The method of claim 1 further comprising amplifying the cDNA fragments to produce amplified cDNA fragments.

22. The method of claim 21 further comprising:
contacting said amplified cDNA fragments with a solid support comprising nucleic acid probes.

23. The method of claim 22 further comprising:
detecting the presence or absence of hybridization of said amplified cDNA fragments to the nucleic acid probes on the solid support.

24. The method of claim 23 wherein the solid support is selected from the group consisting of a nucleic acid probe array, a membrane blot, a microwell, a bead, and a sample tube.

25. The method of claim 1 wherein the RNA sample is further contacted with primer comprising poly dT.

26. The method of claim 23 wherein hybridization is detected by detecting a signal from labeled DNA which is hybridized to the solid support.

27. The method of claim 26 wherein the signal is amplified.

28. A gene expression monitoring system comprising the labeled cDNA fragments of claim 1 and a solid support comprising nucleic acid probes.

29. A gene expression monitoring system comprising the labeled cDNA fragments of claim 21 and a solid support comprising nucleic acid probes.

30. A kit for the detection of nucleic acids, wherein the kit comprises a container, instructions for use, random primers, buffers, a reverse transcriptase, DNase, a terminal transferase and an a solid support comprising nucleic acid probes.

31. A method of detecting one or more isoforms of RNA in an RNA sample comprising:
contacting an RNA sample with random primers under hybridization conditions;
generating cDNA from the RNA sample by extending the random primers with reverse transcriptase to produce cDNA;
degrading the RNA population;
fragmenting the cDNA;
labeling the cDNA fragments;
contacting the labeled cDNA fragments with an array comprising a probe that hybridizes to the complement of a sequence present in a first isoform but absent from a second isoform; and
detecting the presence or absence of hybridization of the labeled cDNA fragments to the array.

32. The method of claim 31 wherein the RNA sample is selected from the group consisting of total RNA, poly(A)⁺ RNA and MRNA.

33. The method of claim 31 wherein the RNA sample is isolated from a eukaryotic cell or tissue.

34. A method of detecting one or more isoforms of RNA in an RNA sample comprising:
contacting an RNA sample with random primers under hybridization conditions;
generating cDNA from the RNA sample by extending the random primers with reverse transcriptase to produce CDNA;
degrading the RNA population;
fragmenting the cDNA;
labeling the cDNA fragments;
contacting the labeled cDNA fragments with an array comprising a probe that hybridizes to the complement of a sequence common to each of the one or more isoforms; and
detecting the presence or absence of hybridization of the labeled cDNA fragments to the array.

35. The method of claim 34 wherein the RNA sample is selected from the group consisting of total RNA, poly(A)⁺ RNA and MRNA.

36. The method of claim 34 wherein the RNA sample is isolated from a eukaryotic cell or tissue.

37. A method of detecting all RNA transcripts of a single gene present in an RNA sample and distinguishing between different transcript isoforms present in said RNA sample comprising:
contacting an RNA sample with random primers under hybridization conditions;
generating cDNA from the RNA sample by extending the random primers with reverse transcriptase to produce cDNA;
degrading the RNA population;
fragmenting the cDNA;
labeling the cDNA fragments;
contacting the labeled cDNA fragments with an array comprising a probe that hybridizes to the complement of a sequence present in each of the one or more isoforms and a probe that hybridizes to the complement of a sequence common to each of the one or more isoforms; and
detecting the presence or absence of hybridization of the labeled cDNA fragments to the array.

38. The method of claim 37 wherein the RNA sample is selected from the group consisting of total RNA, poly(A)⁺ RNA and mRNA.

39. The method of claim 37 wherein the RNA sample is isolated from a eukaryotic cell or tissue.

40. A method of detecting the presence or absence of transcriptional activity from a region of a genome comprising:
obtaining a sample of RNA transcribed from said genome;
contacting said RNA sample with random primers under hybridization conditions;
generating cDNA from the RNA sample by extending the random primers with reverse transcriptase;
degrading the RNA;
fragmenting the cDNA;
labeling the cDNA fragments;
contacting the labeled cDNA fragments with an array comprising probes that hybridize to a plurality of sequences present in said genome; and
detecting the presence or absence of hybridization of the labeled cDNA fragments to the array.
